(19) United States
(12) Patent Application Publication
(10) Pub. No.: US 2026/0345194 A1
(43) Pub. Date: Nov. 5, 2026

(54) CUTTING BALLOON CATHETER

(71) Applicant: Boston Scientific Scimed, Inc., Maple Grove, MN (US)

(72) Inventors: John Kilcooley, Gort (IE); Michelle Hannon, Galway (IE); Dermot Toomey, Ballybrit (IE)

(73) Assignee: Boston Scientific Scimed, Inc., Maple Grove, MN (US)

(21) Appl. No.: 19/667,120

(22) Filed: May 4, 2026

Related U.S. Application Data
(60) Provisional application No. 63/799,940, filed on May 5, 2025.

(51) Int. Cl. A61B 17/3207

(52) U.S. Cl. CPC A61B 17/320725

(57) ABSTRACT

Example medical devices are disclosed. A cutting balloon includes an expandable member having an outer surface, a longitudinal axis, and a first groove extending along the longitudinal axis. Further, the cutting balloon also has a first cutting member having a base portion and a cutting edge, wherein at least a portion of the base portion is positioned within the first groove. Further, the cutting balloon also has an adhesive material disposed within the groove, wherein the adhesive material is configured to bond the cutting member to the expandable member.

### CROSS-REFERENCE TO RELATED APPLICATION

This application claims the benefit of U.S. Patent Application Serial No. 63/799,940, filed May 5, 2025, entitled “CUTTING BALLOON CATHETER”, which is incorporated by reference herein in its entirety.

### TECHNICAL FIELD

The disclosure is directed to angioplasty balloon catheters including one or more cutting members mounted to the balloon. More particularly, the disclosure is directed to a cutting balloon catheter having a reduced outer profile.

### BACKGROUND

Heart and vascular disease are major problems in the United States and throughout the world. Conditions such as atherosclerosis result in blood vessels becoming blocked or narrowed. This blockage can result in lack of oxygenation to the heart and/or circulation to other regions of the body.

Occluded, stenotic, or narrowed blood vessels, as well as native or synthetic arteriovenous dialysis fistulae, may be treated in a recanalization procedure, such as with an angioplasty balloon catheter advanced over a guidewire to an occlusion so that the balloon is positioned across the occlusion. The balloon may then be inflated to enlarge the passageway through the occlusion.

A major obstacle in treating coronary artery disease and/or treating blocked blood vessels or fistulae is re-stenosis or re-narrowing of the passageway through the occlusion subsequent to an angioplasty procedure or other recanalization procedure. Evidence has shown that cracking, cutting or scoring the stenosis with an angioplasty balloon equipped with a cutting members may reduce incidence of re-stenosis. Additionally, cracking, cutting or scoring the stenosis may reduce trauma at the treatment site and/or may reduce the trauma to adjacent healthy tissue. Cutting members may also be beneficial additions to angioplasty procedures when the targeted occlusion is hardened or calcified. Thus, angioplasty balloons equipped with cutting members have been developed to enhance angioplasty treatments.

In some examples, cutting members may be adhesively secured to an angioplasty balloon. Accordingly, it can be appreciated that the cutting members may extend radially away from the outer surface of the balloon, thereby defining an overall profile of the cutting balloon. It can be appreciated that a lower crossing profile may improve the deliverability of the cutting balloon, as a lower overall profile may permit the cutting balloon to penetrate and cross tight lesions compared to alternative cutting balloon configurations. Accordingly, there is an ongoing need for cutting balloons with lower overall profiles to improve the deliverability of the cutting balloon catheter.

### SUMMARY

The disclosure is directed to several alternative designs, materials and methods of manufacturing medical device structures and assemblies, and the use thereof. A cutting balloon includes an expandable member having an outer surface, a longitudinal axis, and a first groove extending along the longitudinal axis. Further, the cutting balloon also has a first cutting member having a base portion and a cutting edge, wherein at least a portion of the base portion is positioned within the first groove. Further, the cutting balloon also has an adhesive material disposed within the groove, wherein the adhesive material is configured to bond the cutting member to the expandable member.

In addition or alternatively to any example described herein, wherein the adhesive material surrounds at least a portion of the base portion.

In addition or alternatively to any example described herein, wherein the base portion includes a curved bottom surface.

In addition or alternatively to any example described herein, wherein the groove includes a curved surface configured to match the curved bottom surface of the base portion.

In addition or alternatively to any example described herein, wherein the groove includes a rectangular cross-sectional shape.

In addition or alternatively to any example described herein, wherein the groove includes a tapered cross-sectional shape.

In addition or alternatively to any example described herein, wherein the groove extends radially inward from the outer surface of the balloon a distance between 0.05 mm and 0.40 mm.

In addition or alternatively to any example described herein, wherein the groove includes a width between 0.015 mm and 0.40 mm.

In addition or alternatively to any example described herein, wherein the cutting edge extends radially away from the outer surface of the balloon, and wherein the cutting edge is free of the adhesive material.

In addition or alternatively to any example described herein, wherein the cutting edge extends radially away from the outer surface of the balloon a distance between 0.10 mm and 0.30 mm.

In addition or alternatively to any example described herein, wherein the adhesive material is disposed within the groove such that an outer facing surface of the adhesive material is substantially flush with the outer surface of the balloon.

In addition or alternatively to any example described herein, wherein the cutting balloon includes a second cutting member positioned on a distal cone of the cutting balloon.

In addition or alternatively to any example described herein, wherein the base portion includes a first lateral edge, a second lateral edge, a first plurality of cutouts extending along the first lateral edge, and a second plurality of cutouts extending along the second lateral edge.

In addition or alternatively to any example described herein, further comprising a second groove extending along the longitudinal axis and a second cutting member having a base portion and a cutting edge, wherein at least a portion of the base portion of the second cutting member is positioned within the second groove.

A cutting balloon includes an expandable member having an outer surface, a longitudinal axis, and a groove extending along the longitudinal axis, the expandable member configured to shift between a first configuration to a second expanded configuration. Further, the cutting balloon also includes a cutting member assembly disposed along the groove of the expandable member, the cutting member assembly including a cutting member having a base portion and a cutting edge, wherein at least a portion of the base portion is disposed within the groove and an adhesive material disposed within the groove, wherein the adhesive material is configured to bond the cutting member to the expandable member as the expandable member shifts between the first configuration and the second configuration.

In addition or alternatively to any example described herein, wherein the adhesive material surrounds at least a portion of the base portion.

In addition or alternatively to any example described herein, wherein the cutting edge extends radially away from the outer surface of the balloon, and wherein the cutting edge is free of the adhesive material.

In addition or alternatively to any example described herein, wherein the cutting edge extends radially away from the outer surface of the balloon a distance between 0.10 mm and 0.30 mm.

In addition or alternatively to any example described herein, wherein the adhesive material is disposed within the groove such that an outer facing surface of the adhesive material is substantially flush with the outer surface of the balloon.

A method of treating a body lumen includes advancing a cutting balloon to a target site in the body lumen. Further, the cutting balloon includes an expandable member having an outer surface, a longitudinal axis, and a first groove extending along the longitudinal axis. Further, the cutting balloon also includes a first cutting member having a base portion and a cutting edge, wherein at least a portion of the base portion is positioned within the first groove. Further, the cutting balloon includes an adhesive material disposed within the groove, wherein the adhesive material is configured to bond the cutting member to the expandable member. Further, the method also includes expanding the expandable member such that the cutting member engages the target site.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following description of various examples in connection with the accompanying drawings, in which:

FIG. 1 is a perspective view of an example medical device;

FIG. 2 is a cross-sectional view of the medical device of FIG. 1 taken along line 2-2 of FIG. 1;

FIG. 3 is a cross-sectional view of the balloon of the medical device of FIG. 1;

FIG. 4 is a cross-sectional view of the medical device of FIG. 1 taken along line 4-4 of FIG. 1;

FIG. 5 is a cross-sectional view of another embodiment of the medical device of FIG. 1;

FIG. 6 is a cross-sectional view of another embodiment of the medical device of FIG. 1;

FIG. 7 is a partial perspective view of an example medical device;

FIG. 8 is a partial perspective view of an example medical device;

FIG. 9 is a partial perspective view of an example medical device;

FIG. 10 is a partial perspective view of a portion of an example medical device;

FIG. 11 is a partial perspective view of a portion of an example medical device; and

FIG. 12 is a partial perspective view of a portion of an example medical device.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification. All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

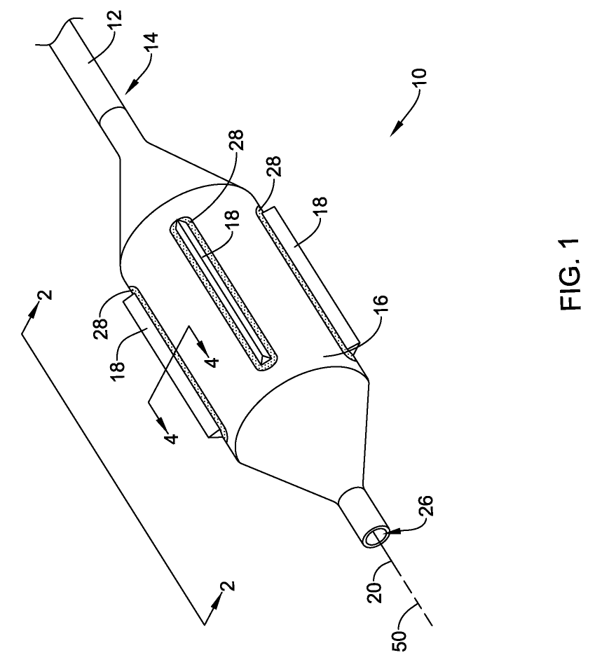

FIG. 1 is a perspective view of an example cutting balloon catheter 10. In some instances, the example cutting balloon catheter 10 may be utilized within a patient’s vasculature in order to treat a stenosis within the vasculature.

The catheter 10 may include a balloon 16 coupled to a distal end region 14 of an outer shaft 12. One or more cutting members or blades 18 may be mounted to the balloon 16. In general, the catheter 10 may be advanced over a guidewire 20, through the vasculature, to a target lesion. Once positioned at the target location in the vasculature, the balloon 16 may be inflated such that the balloon 16 and/or the cutting members 18 exert a radially outward force on the lesion. When the cutting members 18 engage the lesion, they may cut or score the lesion to facilitate enlarging the body lumen in which the lesion is located. It is contemplated that the cutting balloon catheter 10 may be used to treat any lumen in the body, including any peripheral or cardiac lumen where a lesion has developed.

The cutting members 18 may vary in number, position, and arrangement about the balloon 16. For example, the catheter 10 may include one, two, three, four, five, six, seven, eight, nine, ten, eleven, twelve or more cutting members 18 that are disposed at any position along the balloon 16 and in a regular, irregular, or any other suitable pattern. For example, in some embodiments the balloon 16 may include a plurality of cutting members 18 longitudinally aligned at various points around the circumference of the balloon 16. For example, the balloon 16 shown in FIG. 1 illustrates three cutting members 18 aligned along a longitudinal axis 50 of the balloon 16. The cutting members 18 may be symmetrically or asymmetrically spaced around the circumference of the balloon 16.

In some examples, the cutting members 18 may be made from any suitable material such as a metal, metal alloy, polymer, metal-polymer composite, and the like, or any other suitable material. For example, the cutting members 18 may be made from stainless steel, titanium, nickel-titanium alloys, tantalum, iron-cobalt-nickel alloys, or other metallic materials. The cutting members 18 may have a triangular cross-sectional shape, or a square cross-sectional shape, or a rectangular cross-sectional shape, a circular cross-sectional shape or any other desired configuration.

The inflatable balloon 16 may be made from balloon materials including polymers such as polyethylene terephthalate (PET), polytetrafluoroethylene (PTFE), ethylene tetrafluoroethylene (ETFE), polybutylene terephthalate (PBT), polyurethane, polyvinylchloride (PVC), polyether-ester, polyester, polyamide, elastomeric polyamides, polyether block amide (PEBA), as well as other suitable materials, or mixtures, combinations, copolymers thereof, polymer/metal composites, and the like. In some instances, the inflatable balloon 16 may include a single layer of material, whereas in other instances the inflatable balloon 16 may be of a multi-layer construction, including a plurality of layers of materials. For instance, the inflatable balloon 16 may be formed as a co-extrusion or tri-layer extrusion in some instances.

The balloon 16 may be coupled to the distal end region 14 of the outer shaft 12 in any of a number of suitable ways. For example, the balloon 16 may be adhesively or thermally bonded to the outer shaft 12.

The balloon 16 may be configured so that the inflatable balloon 16 includes one or more “wings” or wing-shaped regions when the inflatable balloon 16 is deflated. In some instances, the wings may be configured so that the cutting members 18 can be positioned at the inward-most positions of the deflated inflatable balloon 16, with the wings of the balloon folds positioned between adjacent cutting members 18. This arrangement may minimize the crossing profile of the folded inflatable balloon 16 and reduce the exposure of the cutting members 18 to the blood vessel during delivery of the inflatable balloon 16 to the lesion.

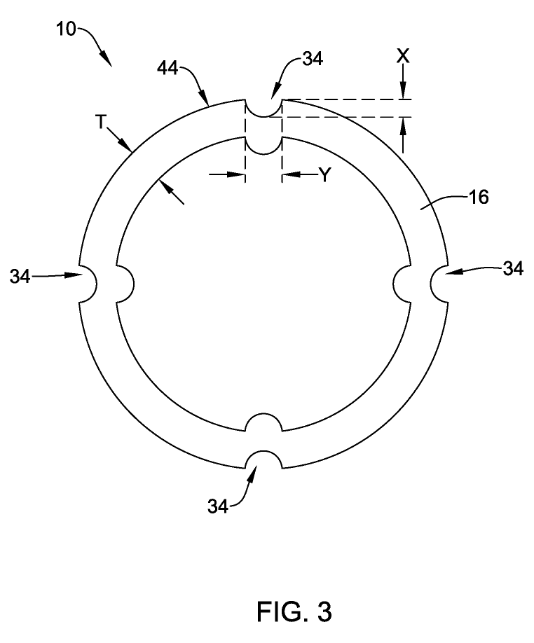

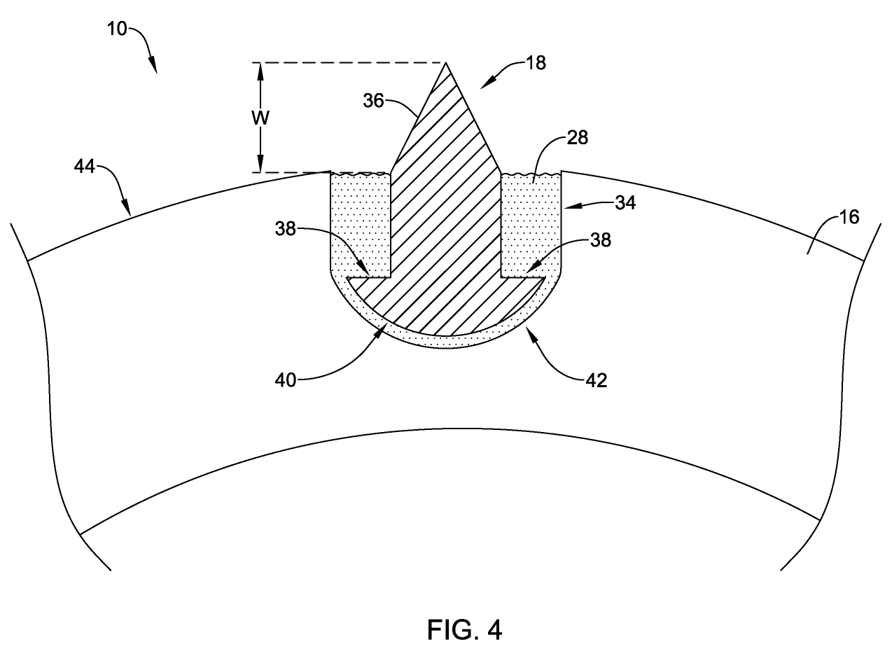

The cutting members 18 may be coupled to the inflatable balloon 16 using a variety of techniques. For example, each of the cutting members 18 may be secured to the balloon 16 by adhesively bonding at least a portion of the cutting member 18 within a groove 34 (shown in FIGS. 3-4) formed within the wall of the balloon 16. It can be appreciated that configuring the balloon 16 to include grooves 34 within which a portion of the cutting members 18 are positioned may reduce the overall outer profile (e.g., maximum outer diameter) of the cutting balloon catheter 10 as compared to other catheters that do not include a similar construction.

Further, in some examples, each blade member 18 may include a base portion that may be at least partially embedded in an adhesive material 28 (e.g., polymeric adhesive) that may itself be secured within a groove 34 (shown in FIGS. 3-4) formed within the wall of the inflatable balloon 16. As illustrated in FIG. 1, each of the cutting members 18 may be attached within its own respective groove 34 within the balloon using an adhesive 28. As shown, each groove 34 may be spaced from one another, thereby spacing each cutting member 18 away from one another around the circumference of the balloon 16.

In some examples, the adhesive material 28 may include a high tensile strength, UV cured, medical grade adhesive with low ductility such as Loctite® 3943, Loctite® 3321 or other similar adhesive materials.

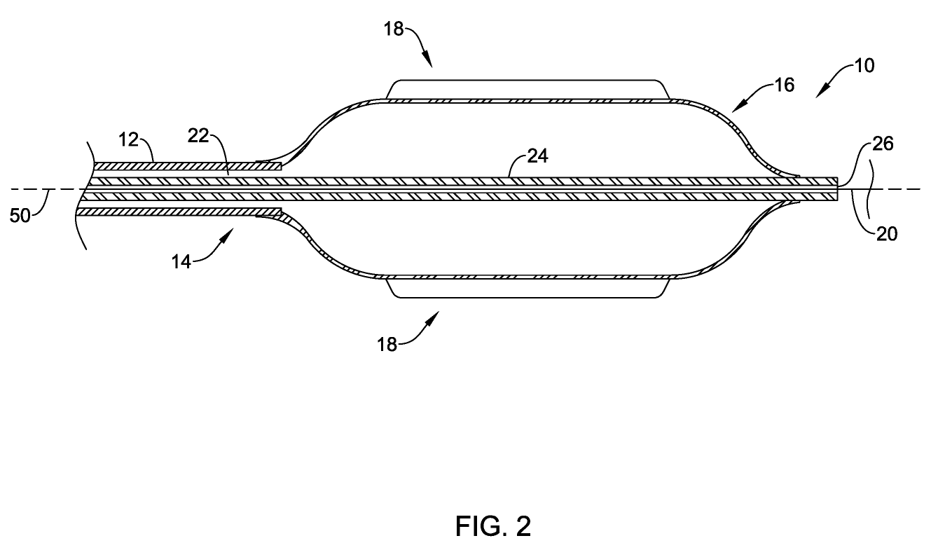

FIG. 2 is a partial cross-sectional side view of the cutting balloon catheter 10 taken along line 2-2 of FIG. 1. FIG. 2 illustrates that the catheter 10 may include an inner shaft 24 disposed coaxially and extending within at least a portion of an inner lumen of the outer shaft 12. The outer shaft 12 and/or the inner shaft 24 may be made of a variety of materials including polymers, metals, metal alloys, metal-polymer composites or any other suitable materials. In some embodiments, the inner shaft 24 and outer shaft 12 may or may not be secured to one another. In other examples, the inner shaft 24 may follow an inner wall of the outer shaft 12 or otherwise be disposed adjacent the inner wall of the outer shaft 12.

The inner shaft 24 may include an inner lumen 26. In at least some embodiments, the inner lumen 26 of the inner shaft 24 may be a guidewire lumen for receiving the guidewire 20 therethrough. Accordingly, the catheter 10 can be advanced over the guidewire 20 to a desired target location in the body. In some examples, the guidewire lumen 26 may extend along the entire length of the inner shaft 24 in an “over-the-wire” catheter configuration. Alternatively, the guidewire lumen 26 may extend along only a portion of the inner shaft 24 in a “single-operator-exchange” or “rapid-exchange” catheter configuration.

The catheter 10 may also include an inflation lumen 22 that may be used, for example, to transport inflation media to and from the inflatable balloon 16 to selectively inflate and/or deflate the inflatable balloon 16. The location and position of the inflation lumen 22 may vary, depending on the configuration of the outer shaft 12 and the inner shaft 24. For example, when the outer shaft 12 surrounds the inner shaft 24, the inflation lumen 22 may be defined within the space between the outer shaft 12 and the inner shaft 24. In instances in which the outer shaft 12 is disposed alongside the inner shaft 24, the inflation lumen 22 may be a lumen of the outer shaft 12. It can further be appreciated that, in some examples, the catheter shaft 12 may be constructed as a “multi-lumen” shaft, in which a single shaft includes multiple lumen extending therein, rather than having a distinct inner shaft 24 extending within a lumen of an outer shaft 12, as described above.

FIG. 3 illustrates a cross-section of the balloon 16 showing a plurality of grooves 34 positioned radially inward from an outer surface 44 of the balloon 16. For clarity purposes, the cutting member 18 and adhesive 28 described herein has been omitted from the cross-section show in FIG. 3. Referring to FIG. 1, it can be appreciated that each groove 34 may extend longitudinally along the balloon 16, whereby each groove 34 is configured to accept a portion of a cutting member 18 and an adhesive material 28 utilized to bond the cutting member 18 to the balloon 16.

FIG. 3 illustrates that, in some examples, each groove 34 may be generally U-shaped. However, as will described in greater detail below, a variety of profiles are contemplated for the shape of each groove 34. For example, the cross-sectional profile of each groove 34 may be generally square-shaped, rectangular-shaped, triangular-shaped, ovular-shaped, circular-shaped, star-shaped, or any other suitable cross-sectional shape.

FIG. 3 further illustrates the balloon 16 including four grooves 34 circumferentially spaced approximately 90 degrees from one another. It is contemplated that the balloon 16 may include 1, 2, 3, 4, 5, 6, 7, 8 or more grooves 34. It is further contemplated that the grooves 34 may be spaced equidistant from one another around the circumference of the balloon 16. In other examples, the grooves 34 may be variably spaced from one another around the circumference of the balloon 16.

FIG. 3 illustrates that each groove 34 may extend radially inward from an outer surface 44 (e.g., the depth of the groove 34) of the balloon a distance “X”. In some instances, the distance X may be about 0.05 mm to about 0.40 mm, or about 0.075 mm to about 0.35 mm, or about 0.10 mm to about 0.30 mm, or about 0.15 mm to about 0.25 mm. Further, FIG. 3 illustrates that each groove 34 may include a width “Y”. In some instances, the width Y may be about 0.015 mm to about 0.40 mm, or about 0.020 mm to about 0.35 mm, or about 0.10 mm to about 0.30 mm, or about 0.15 mm to about 0.25 mm. FIG. 3 further illustrates that the balloon 16 may include a wall thickness “T”. In some examples, the wall thickness T may be about 0.008 mm to about 0.050 mm, or about 0.015 mm to about 0.040 mm, or about 0.025 mm to about 0.035 mm.

FIG. 3 illustrates that, in some examples, the thickness T of the balloon 16 may remain substantially constant along the circumference of the balloon 16. However, in other examples, the thickness T of the balloon 16 may vary along the along the circumference of the balloon 16. For example, in some instances the inner surface of the balloon 16 may be a continuous surface in which the inner radius extending from a longitudinal axis of the balloon 16 to the inner surface of the balloon 16 may be constant. Accordingly, at positions adjacent the groove 34, the wall thickness T may be thinner than other positions along the balloon 16. In other examples, such as that shown in FIG. 3, the inner surface of the balloon 16 may be a continuous surface in which the radius from a longitudinal axis of the balloon 16 to the inner surface of the balloon 16 varies. Accordingly, the wall thickness T of the balloon 16 may remain substantially constant along the circumference of the balloon 16.

FIG. 4 illustrates a cross-sectional view taken along line 4-4 of FIG. 1. FIG. 4 illustrates cutting member 18 positioned in a groove 34 extending radially inward from an outer surface 44 of the balloon 16. FIG. 4 further illustrates that a portion of the cutting member 18 may be fully or partially positioned within an adhesive material 28. In some examples, a portion of the cutting member 18 may be fully or partially embedded within the adhesive material 28, whereby the adhesive material 28 surrounds a portion of the outer surface of the cutting member 18.

As illustrated in FIG. 4, the cutting member 18 may include a base portion 40 and a cutting edge 36 opposite the base portion 40. It can be appreciated from FIG. 4 that the cutting edge 36 of the cutting member 18 may extend radially away from the outer surface 44 of the balloon 16 a distance “W”. In some examples, the distance W may be about 0.10 mm to about 0.40 mm, or about 0.15 mm to about 0.35 mm, or about 0.12 mm to about 0.30 mm. As discussed herein, configuring the balloon 16 to include grooves 34 within which a portion of the cutting members 18 are positioned may reduce the overall outer profile (e.g., maximum outer diameter) of the cutting balloon catheter 10 (as compared to other catheters that do not include a similar construction) because only the cutting edge 36 (versus the entire height of the cutting member 18) of the cutting member 18 may extend radially away from the outer surface 44 of the balloon 16.

FIG. 4 illustrates that the base portion 40 may include two securement surfaces 38 which may be configured to provide additional contact surface area along which the adhesive material 28 may bond. For example, FIG. 4 illustrates that after the cutting member 18 is positioned within the groove 34, the adhesive material 28 may be inserted into the groove 34 such that it surrounds and encapsulates a portion of the cutting member 18, including the base portion 40. After being inserted into the groove 34, the adhesive material may cure, thereby chemically and/or mechanically bonding the cutting member 18 to the balloon 16. In some examples, the adhesive material 28 may be inserted into the groove 34 such that the top surface of the adhesive material 28 may be substantially flush with the outer surface 44 of the balloon 16. In other examples, the adhesive material 28 may extend radially outward of or beyond the outer surface 44 of the balloon 16 such that the outer extent of the adhesive material 28 is raised relative to the outer surface 44 of the balloon 16. In yet other examples, the adhesive material 28 may extend radially inward of or below the outer surface 44 of the balloon 16, such that the outer extent of the adhesive material 28 is recessed radially inward of the outer surface 44 of the balloon 16.

FIG. 4 further illustrates that the base portion 40 of the cutting member 18 may include a profile (e.g., cross-sectional profile) which substantially matches a profile 42 (e.g., cross-sectional profile) of the bottom of the groove 34. For example, FIG. 4 illustrates the base portion 40 of the cutting member 18 may include a substantially curved profile which matches a curved profile 42 of the bottom of the groove 34.

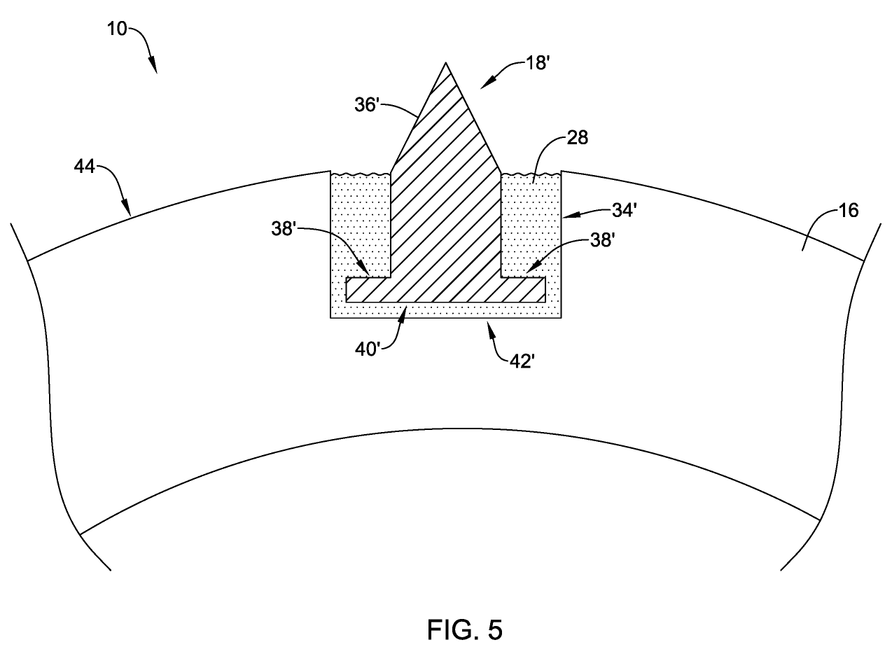

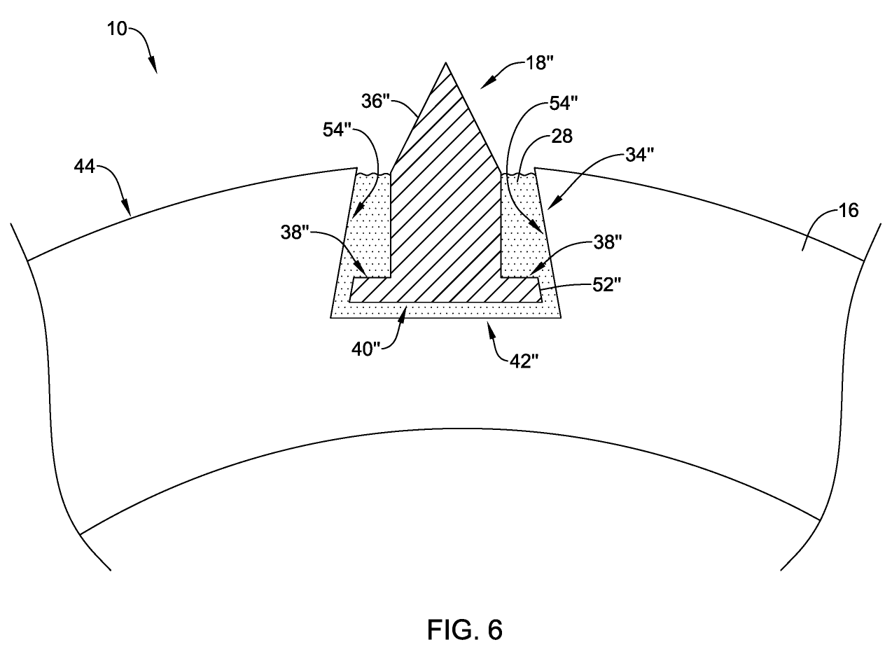

FIGS. 5-6 illustrate alternative configurations for the shape of the base portion 40 and the bottom of the groove 34 of the cutting member 18 and the groove 34 illustrated in FIG. 4.

For example, FIG. 5 illustrates that the base portion 40′ of the cutting member 18′ may include a substantially rectangular or square profile (e.g., rectangular or square cross-sectional profile) which matches a rectangular or square profile 42′ (e.g., rectangular or square cross-sectional profile) of the bottom of the groove 34′. FIG. 5 illustrates that the base portion 40′ may include two securement surfaces 38′ which may be configured to provide additional contact surface area along which the adhesive material 28 may bond. For example, FIG. 5 illustrates that after the cutting member 18′ is positioned within the groove 34′, the adhesive material 28 may be inserted into the groove 34′ such that it surrounds and encapsulates a portion of the cutting member 18′, including the base portion 40′. After being inserted into the groove 34′, the adhesive material may cure, thereby chemically and/or mechanically bonding the cutting member 18′ to the balloon 16.

In other examples, FIG. 6 illustrates the base portion 40′′ of the cutting member 18′′ may include a substantially tapered profile (e.g., tapered cross-sectional profile) which matches a tapered profile 42′′ (e.g., tapered cross-sectional profile) of the bottom of the groove 34′′. For example, FIG. 6 illustrates that the base portion 40′′ may include tapered surfaces 52′′ which may be configured to match tapered surfaces 54′′ of the groove 34′′. Additionally, FIG. 6 illustrates that the base portion 40′′ may include two securement surfaces 38′ which may be configured to provide additional contact surface area along which the adhesive material 28 may bond. For example, FIG. 6 illustrates that after the cutting member 18′′ is positioned within the groove 34′′, the adhesive material 28 may be inserted into the groove 34′′ such that it surrounds and encapsulates a portion of the cutting member 18′′, including the base portion 40′′. After being inserted into the groove 34′′, the adhesive material may cure, thereby chemically and/or mechanically bonding the cutting member 18′′ to the balloon 16.

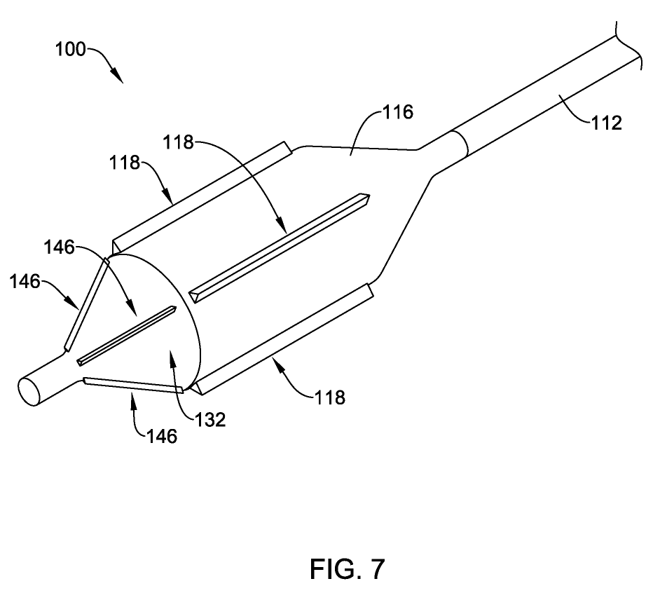

FIG. 7 illustrates another example cutting balloon catheter 100. The cutting balloon catheter 100 may be similar in form and function to the cutting balloon catheter 10 described herein. For example, the cutting balloon catheter 100 may include a balloon 116 coupled to an outer shaft 112. Further, the cutting balloon catheter 100 may include one or more cutting members 118 attached to the balloon 116 using techniques similar to that illustrated and described with respect to the cutting members 18 and balloon 16 of FIG. 1. For example, the cutting members 118 may be chemically and/or mechanically bonded within grooves of the balloon 116 using an adhesive material similar in form and function to the adhesive material 28 described with respect to FIG. 1.

FIG. 7 further illustrates that, in some examples, the cutting balloon catheter 100 may include one or more cutting members 146 positioned along a distal cone 132 of the balloon 116. The cutting members 146 may be attached along the distal cone 132 of the balloon 116 using techniques similar to that illustrated and described with respect to the cutting members 18 and balloon 16 of FIG. 1. For example, the cutting members 146 may be chemically and/or mechanically bonded within grooves positioned along the distal cone 132 of the balloon 116 using an adhesive material similar in form and function to the adhesive material 28 described with respect to FIG. 1. The cutting members 146 may vary in number, position, and arrangement about the distal cone 132 of the balloon 116. For example, the catheter 100 may include one, two, three, four, five, six, seven, eight, nine, ten, eleven, twelve or more cutting members 146 that are disposed at any position along the distal cone 132 of the balloon 116 and in a regular, irregular, or any other suitable pattern. The cutting members 146 may be symmetrically or asymmetrically spaced around the circumference of the distal cone 132 of the balloon 116.

It can be appreciated that positioning cutting members 146 along the distal cone 132 of the balloon 116 may provide the balloon 116 with a forward-facing cutting advantage. For example, the cutting members 146 may provide the catheter 100 with a plurality of leading cutting edges which may be beneficial in piercing or gaining initial entry into a tight lesion. Further, the cutting members 146 may be utilized to gain an initial purchase into a tight lesion despite the balloon 116 being in a wrapped configuration. It can be further appreciated that once the distal cone 132 has been fully or partially inserted into the lesion, the balloon 116 may be inflated whereby the cutting members 146 may be begin the initial cracking, cutting or scoring the lesion with the balloon 116. The balloon 116 may then be progressively inserted into the lesion whereby the cutting members 118 and balloon 116 may be used to further expand the lesion.

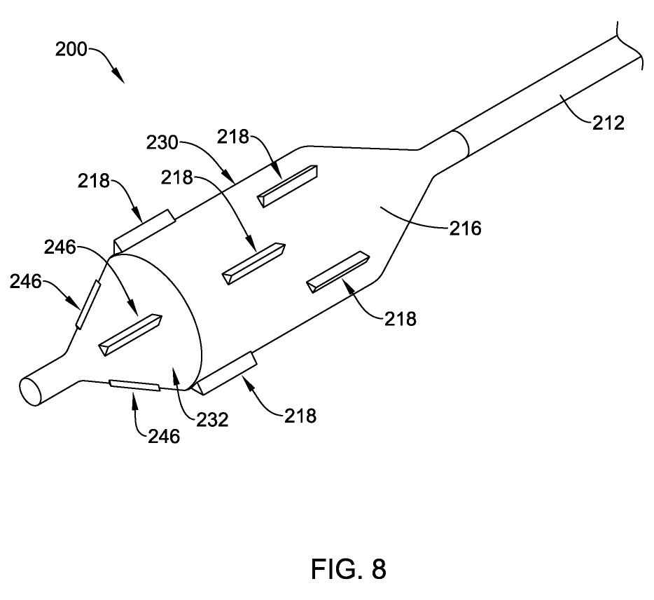

FIG. 8 illustrates another example cutting balloon catheter 200. The cutting balloon catheter 200 may be similar in form and function to the cutting balloon catheters 10, 100 described herein. For example, the cutting balloon catheter 200 may include a balloon 216 coupled to an outer shaft 212. Further, the cutting balloon catheter 200 may include one or more cutting members 218 attached to the balloon 216 using techniques similar to that illustrated and described with respect to the cutting members 18 and balloon 16 of FIG. 1. For example, the cutting members 218 may be chemically and/or mechanically bonded within grooves of the balloon 216 using an adhesive material similar in form and function to the adhesive material 28 described with respect to FIG. 1.

Further, FIG. 8 illustrates that the cutting members 218 of the catheter 200 may be relative shorter in length compared to the cutting members 218 illustrated with respect to FIG. 1. Further, FIG. 8 illustrates that the catheter 200 may include one, two, three, four, five, six, seven, eight, nine, ten, eleven, twelve or more cutting members 218 that are disposed at any position along a body 230 of the balloon 116 and in a regular, irregular, or any other suitable pattern such as the staggard pattern illustrated in FIG. 8. The cutting members 218 may be symmetrically or asymmetrically spaced around the circumference of the balloon 216.

FIG. 8 further illustrates that, in some examples, the cutting balloon catheter 200 may include one or more cutting members 246 positioned along a distal cone 232 of the balloon 216. The cutting members 246 may be attached along the distal cone 232 of the balloon 216 using techniques similar to that illustrated and described with respect to the cutting members 18 and balloon 16 of FIG. 1. For example, the cutting members 246 may be chemically and/or mechanically bonded within grooves positioned along the distal cone 232 of the balloon 216 using an adhesive material similar in form and function to the adhesive material 28 described with respect to FIG. 1. The cutting members 246 may vary in number, position, and arrangement about the distal cone 232 of the balloon 216. For example, the catheter 200 may include one, two, three, four, five, six, seven, eight, nine, ten, eleven, twelve or more cutting members 246 that are disposed at any position along the distal cone 232 of the balloon 216 and in a regular, irregular, or any other suitable pattern. The cutting members 246 may be symmetrically or asymmetrically spaced around the circumference of the distal cone 232 of the balloon 216.

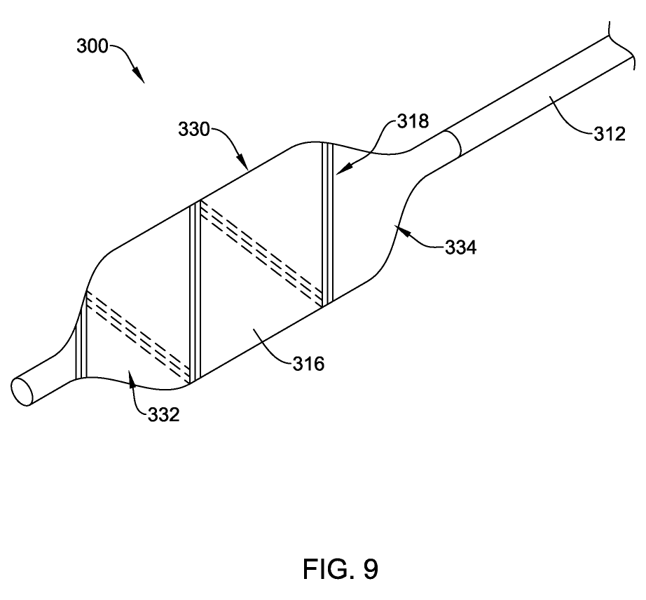

FIG. 9 illustrates another example cutting balloon catheter 300. The cutting balloon catheter 300 may be similar in form and function to the cutting balloon catheters 10, 100, 200 described herein. For example, the cutting balloon catheter 300 may include a balloon 316 coupled to an outer shaft 312.

Further, FIG. 9 illustrates that the cutting balloon catheter 300 may include one or more cutting members 318 attached to the balloon 316 using techniques similar to that illustrated and described with respect to the cutting members 18 and balloon 16 of FIG. 1. For example, the cutting member 318 may be chemically and/or mechanically bonded within a groove of the balloon 316 using an adhesive material similar in form and function to the adhesive material 28 described with respect to FIG. 1. FIG. 3 illustrates that the cutting member 318 may extend around the balloon 316 in a spiral configuration. It can be appreciated that the cutting member 318 may extend in a spiral configuration along any portion of a distal cone 332, a proximal cone 334 and/or a body 330 of the balloon 316.

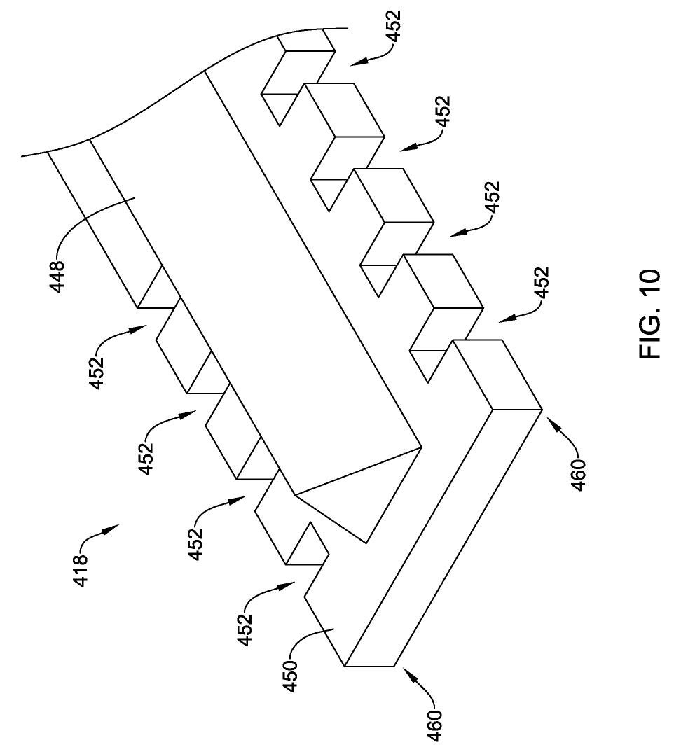

FIG. 10 illustrates a portion of an example cutting member 418. The cutting member 418 may be similar in form and function to the cutting member 18 described herein. FIG. 10 illustrates that the cutting member 418 may include a cutting edge 448 coupled to a base portion 450. The base portion 450 may include a first lateral edge 460 extending along a first side of the base portion 450 and a second lateral edge 460 extending along a second, opposite side of the base portion 450. The first and second lateral edges 460 may extend between first and second ends of the base portion 450, and in some instances, the first and second lateral edges 460 may extend substantially parallel to the cutting member 418, such as substantially parallel to the cutting edge 448 of the cutting member 418. Further, FIG. 10 illustrates that the base portion 450 may include one or more cutouts 452 extending inward from the lateral edges 460 of the base portion 450 toward the cutting member 418. Thus, the cutouts 452 formed along the first lateral edge 460 may extend toward the opposing second lateral edge 460, and likewise, the cutouts 452 formed along the second lateral edge 460 may extend toward the opposing first lateral edge 460. It can be appreciated that the cutouts 452 may provide additional surface area for the adhesive material 28 (shown in FIG. 1) to contact when chemically and/or mechanically bonding the cutting member 418 to the balloon 416.

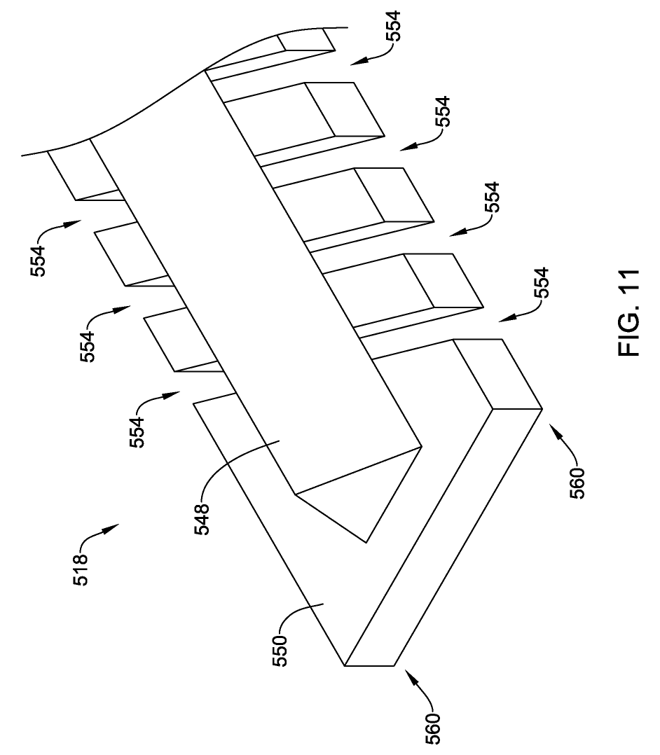

FIG. 11 illustrates a portion of an example cutting member 518. The cutting member 518 may be similar in form and function to the cutting member 18 described herein. FIG. 11 illustrates that the cutting member 518 may include a cutting edge 548 coupled to a base portion 550. The base portion 550 may include a first lateral edge 560 extending along a first side of the base portion 550 and a second lateral edge 560 extending along a second, opposite side of the base portion 550. The first and second lateral edges 560 may extend between first and second ends of the base portion 550, and in some instances, the first and second lateral edges 560 may extend substantially parallel to the cutting member 518, such as substantially parallel to the cutting edge 548 of the cutting member 518. Further, FIG. 11 illustrates that the base portion 550 may include one or more channels 554 extending inward from the lateral edges 560 of the base portion 550 toward the cutting member 518. Thus, the channels 554 formed along the first lateral edge 560 may extend toward and/or to the opposing second lateral edge 560, and likewise, the channels 554 formed along the second lateral edge 560 may extend toward or to the opposing first lateral edge 560. It can be appreciated that the channels 554 may provide additional surface area for the adhesive material 28 (shown in FIG. 1) to contact when chemically and/or mechanically bonding the cutting member 518 to the balloon 516.

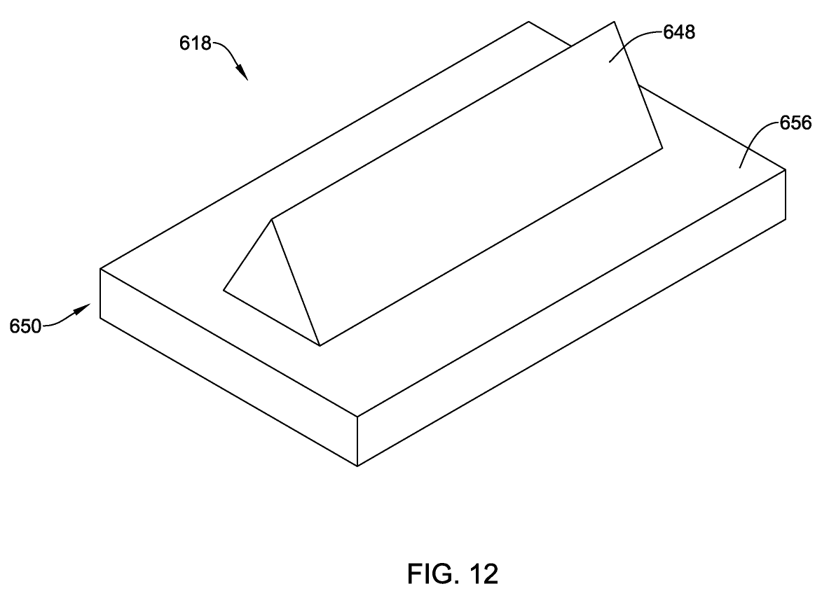

FIG. 12 illustrates a portion of an example cutting member 618. The cutting member 618 may be similar in form and function to the cutting member 18 described herein. FIG. 12 illustrates that the cutting member 618 may include a cutting edge 648 coupled to a base portion 650. Further, FIG. 12 illustrates that the base portion 650 may include a bonding surface 656 configured to provide an extended or generally wider contact surface for which the adhesive material 28 (shown in FIG. 1) to contact when chemically and/or mechanically bonding the cutting member 618 to the balloon 616.

In some embodiments, the components of the cutting balloon catheter 10 and variations thereof described herein may be made from a metal, metal alloy, polymer, a metal-polymer composite, ceramics, combinations thereof, and the like, or other suitable material. A non-exhaustive list of some example materials are listed below.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure’s scope is defined in the language in which the appended claims are expressed.

What is claimed is:

1. A cutting balloon, comprising:
an expandable member having an outer surface, a longitudinal axis, and a first groove extending along the longitudinal axis;
a first cutting member having a base portion and a cutting edge, wherein at least a portion of the base portion is positioned within the first groove; and
an adhesive material disposed within the groove, wherein the adhesive material is configured to bond the cutting member to the expandable member.

2. The cutting balloon of claim 1, wherein the adhesive material surrounds at least a portion of the base portion.

3. The cutting balloon of claim 1, wherein the base portion includes a curved bottom surface.

4. The cutting balloon of claim 3, wherein the groove includes a curved surface configured to match the curved bottom surface of the base portion.

5. The cutting balloon of claim 1, wherein the groove includes a rectangular cross-sectional shape.

6. The cutting balloon of claim 1, wherein the groove includes a tapered cross-sectional shape.

7. The cutting balloon of claim 1, wherein the groove extends radially inward from the outer surface of the balloon a distance between 0.05 mm and 0.40 mm.

8. The cutting balloon of claim 7, wherein the groove includes a width between 0.015 mm and 0.40 mm.

9. The cutting balloon of claim 1, wherein the cutting edge extends radially away from the outer surface of the balloon, and wherein the cutting edge is free of the adhesive material.

10. The cutting balloon of claim 9, wherein the cutting edge extends radially away from the outer surface of the balloon a distance between 0.10 mm and 0.30 mm.

11. The cutting balloon of claim 1, wherein the adhesive material is disposed within the groove such that an outer facing surface of the adhesive material is substantially flush with the outer surface of the balloon.

12. The cutting balloon of claim 1, wherein the cutting balloon includes a second cutting member positioned on a distal cone of the cutting balloon.

13. The cutting balloon of claim 1, wherein the base portion includes a first lateral edge, a second lateral edge, a first plurality of cutouts extending along the first lateral edge, and a second plurality of cutouts extending along the second lateral edge.

14. The cutting balloon claim 1, further comprising a second groove extending along the longitudinal axis and a second cutting member having a base portion and a cutting edge, wherein at least a portion of the base portion of the second cutting member is positioned within the second groove.

15. A cutting balloon, comprising;
an expandable member having an outer surface, a longitudinal axis, and a groove extending along the longitudinal axis, the expandable member configured to shift between a first configuration to a second expanded configuration; and
a cutting member assembly disposed along the groove of the expandable member, the cutting member assembly including:
a cutting member having a base portion and a cutting edge, wherein at least a portion of the base portion is disposed within the groove; and
an adhesive material disposed within the groove, wherein the adhesive material is configured to bond the cutting member to the expandable member as the expandable member shifts between the first configuration and the second configuration.

16. The cutting balloon of claim 15, wherein the adhesive material surrounds at least a portion of the base portion.

17. The cutting balloon of claim 15, wherein the cutting edge extends radially away from the outer surface of the balloon, and wherein the cutting edge is free of the adhesive material.

18. The cutting balloon of claim 15, wherein the cutting edge extends radially away from the outer surface of the balloon a distance between 0.10 mm and 0.30 mm.

19. The cutting balloon of claim 15, wherein the adhesive material is disposed within the groove such that an outer facing surface of the adhesive material is substantially flush with the outer surface of the balloon.

20. A method of treating a body lumen, the method comprising:
advancing a cutting balloon to a target site in the body lumen, the cutting balloon including:
an expandable member having an outer surface, a longitudinal axis, and a first groove extending along the longitudinal axis;
a first cutting member having a base portion and a cutting edge, wherein at least a portion of the base portion is positioned within the first groove; and
an adhesive material disposed within the groove, wherein the adhesive material is configured to bond the cutting member to the expandable member;
expanding the expandable member such that the cutting member engages the target site.
